(19) United States
(12) Patent Application Publication
(10) Pub. No.: US 2002/0077362 A1
(43) Pub. Date: Jun. 20, 2002

(54) Higher unsaturated fatty acid composition

(75) Inventors: Insu Lee, Bethesda, MD (US); Unjoo Kim, Daejon City (KR)

Correspondence Address: PERKINS COIE LLP, P.O. BOX 2168, MENLO PARK, CA, 94026, US

(73) Assignee: SUNDORY CO., LTD.

(21) Appl. No.: 10/039,851

(22) Filed: Jan. 3, 2002

Related U.S. Application Data
(62) Division of application No. 09/499,693, filed on Feb. 8, 2000.

(30) Foreign Application Priority Data
Mar. 10, 1999 (KR) 1999-7943
Jun. 7, 1999 (KR) 1999-21017

(51) Int. Cl. A23D 9/00; A61K 31/202

(52) U.S. Cl. 514/560; 426/601

(57) ABSTRACT

A composition that makes it possible to take DHA efficiently. The composition contains linoleic fatty acid (n-6, 18:2) and α-linolenic fatty acid (n-6, 18:2) so that the weight ratio of the linoleic fatty acid to the α-linolenic fatty acid is 0.05 to 7.5.

### BACKGROUND OF THE INVENTION

This invention relates to a higher unsaturated fatty acid composition comprising linoleic acid and α-linolenic acid.

Docosahexaenoic acid (hereinafter “DHA”) is a main higher unsaturated fatty acid present in the brains of mammals. It is well-known that DHA plays an important role in visual identification, recognition and learning faculty and memory.

DHA is involved in the synthesis of cholesterol, blood clotting inhibition, aging and cancer prevention. Also, it has been recently found out that DHA is beneficial for the treatment of cardiovascular ailments, arthritis rheumatica and asthma and other lung diseases.

One has to take DHA and other higher unsaturated fatty acids from foods because they are not produced in human body. With a rapid increase in old people population, the number of people suffering from age-related illnesses such as senile dementia is increasing. Such age-related diseases are closely related to the function of brain. Thus a method is desired to strengthen the brain function by replenishing DHA from foods.

Synthesis of DHA is derived from α-linolenic acid through the following five enzyme reaction steps, desaturase, 18:4, octadecateranoic acid, elongase, 20:4, arachidonic acid, desaturase, 20:5, eicosapentanoic acid, elongase, 22:5, doscosapentanoic acid, desaturase, 22:6, docosahexanoic acid.

Daily amount of DHA required for an adult is considered to be 300-400 mg per day. Since a fatty acid having 18 or more carbon atoms is considered to be formed from linoleic fatty acid (n-6, 18:2) and α-linolenic fatty acid (n-3, 18:3) both having 18 carbon atoms, for the production of docosahe xaenoic acid (DHA), linoleic fatty acid (n-6, 18:2) and α-linolenic fatty acid (n-3, 18:3) are considered to be essential fatty acids. But there is few method known for taking a required amount of DHA efficiently.

No scientific data exist on the influence of DHA on the function of the brain and eyes. Thus, nothing is known about how DHA can be taken most efficiently.

An object of this invention is to provide a method for making it possible to take DHA efficiently.

### SUMMARY OF THE INVENTION

According to this invention, there is provided a higher unsaturated fatty acid composition comprising linoleic fatty acid (n-6, 18:2) and α-linolenic fatty acid (n-3, 18:3), the weight ratio of said linoleic fatty acid (n-6, 18:2) to said α-linolenic fatty acid (n-3, 18:3) being from 0.05 to 7.5.

It was found out that by adjusting the weight ratio of linoleic fatty acid (n-6, 18:2) to α-linolenic fatty acid (n-3, 18:3) to the above value, DHA is synthesized most efficiently from this composition in the body, particularly in brain, so that this composition strengthens the cognitive and learning faculty and memory.

Other features and objects of the present invention will become apparent from the following description made with reference to the accompanying drawings, in which:

### BRIEF DESCRIPTION OF THE DRAWINGS

FIGS. 1 and 2 are graphs showing the relationship between the dosage of the higher unsaturated fatty acid composition and the cerebral DHA concentration;

FIG. 3 is a graph showing the relationship between the dosage of the higher unsaturated fatty acid composition and the cognitive or learning ability in the Morris maze test; and

FIG. 4 is a graph showing the relationship between the dosage of the higher unsaturated fatty acid composition and the memory in the Morris maze test.

### DETAILED DESCRIPTION OF THE PREFERRED EMBODIMENT

The higher unsaturated fatty acid composition according to the present invention comprises linoleic fatty acid (n-6, 18:2)(hereinafter simply “linoleic acid”) and α-linolenic fatty acid (n-3, 18:3) (hereinafter α-linolenic acid).

Linoleic acid and α-linolenic acid can be extracted from various vegetable and animal oils, such as flax powder, flaxseed meal, flaxseed oil, other flax oils, and Perilla oil. They may be used separately or mixed together. The linoleic and α-linolenic acids used in the present invention need not be refined ones. An intended higher unsaturated fatty acid composition may be prepared by adding oils containing linoleic and α-linolenic acid to foods containing higher unsaturated fatty acids such as food for livestock so that the contents of linoleic and α-linolenic acid will be predetermined values.

The weight ratio of linoleic acid to α-linolenic acid is preferably 0.05-7.5, more preferably 0.05-2.0. If this ratio is below 0.05, the daily supply of linoleic acid would not reach the required minimum. This would reduce the amount of DHA in the brain. It will also decrease if the above ratio is over 7.5. It will increase if the above ratio is between 0.05 and 2.0.

The higher unsaturated fatty acid composition according to this invention may be taken alone as a health food, or may be added to or mixed with other foods.

The higher unsaturated fatty acid composition according to this invention increases the amount of DHA in the brain of an animal to which this composition is administered, thus strengthening its cognitive power, learning power and memory. For this purpose, the daily dose of this composition is preferably 9-18 grams per 60 kg body weight (for an adult).

For example, it is necessary to take ⅓ of the daily requirement at each meal (i.e. at least 3 grams per meal for an adult weighing 60 kg), or eight capsules each weighing 0.4 gram at each meal. For this purpose, the composition of the present invention may be added to various foods (broiled or cooked), soy oil as a salad oil, dairy products, capsules, bakeries, cereals, soy milk, etc.

If pregnant women or newborns are given this composition, or if adults take this composition by at least 9-18 grams daily per 60 kg body weight, synthesis of DHA in the brain will become maximum at birth, weaning, or at coming of age. Thus, if the higher unsaturated fatty acid composition is taken continuously not only during pregnancy but up to five years old, it is possible to beef up the DHA concentration in the young brain. The composition can be taken in the form of practically all kinds of foods including snacks, baked foods, salad dressings, soy oil, dairy products, bread, soymilk, etc. In the case of pregnant women and newborns, if the average daily intake of foods of an adult weighing 60 kg is 1.1 kg, they should take 9-18 grams of the higher unsaturated fatty acid composition. For this purpose, they may take 3 grams of this composition at each meal, or take 8-10 capsules each containing 0.4 gram of this composition, three times a day.

The present composition can thus be used as a cognitive power and memory improver. By adding to foods such a composition, foods can be obtained which can increase the cognitive power and memory.

This higher unsaturated fatty acid composition can be used to improve recognition ability (learning ability) and memory.

If used as a recognition ability (learning ability), memory improver, the composition should be added to foods at the rate of 3 to 5% of the total food weight. For an adult weighing 60 kg, this composition should be preferably given by 3 to 6 grams at each meal, a total of 9 to 18 grams a day.

### [Examples]

[Manufacture of higher unsaturated fatty acid compositions]

Flaxseed, repseed and perilla oils were ground, subjected to fatty acid analysis, mixed together to prepare higher unsaturated fatty acid compositions so that the weight ratio of linoleic acid to α-linolenic acid would be 2, 7.5, 10 and 16. There specimens were given to rats by mixing in their feed so that the content of the higher unsaturated fatty acid composition will be 3 to 5% with respect to the feed.

[Examples of the Invention and Comparative Examples]

The above-mentioned foods were given to pregnant rats every day from the first day of pregnancy to delivery so that the dose of the composition would be 0.15 gram per kilogram of body weight. Newborn rats were also given these feeds for eight weeks. These young rats were then subjected to cerebral fatty acid analysis and learning power/memory retention ability tests.

The learning power/memory retention ability test were conducted by the following Morris maze test and statistically strengthened.

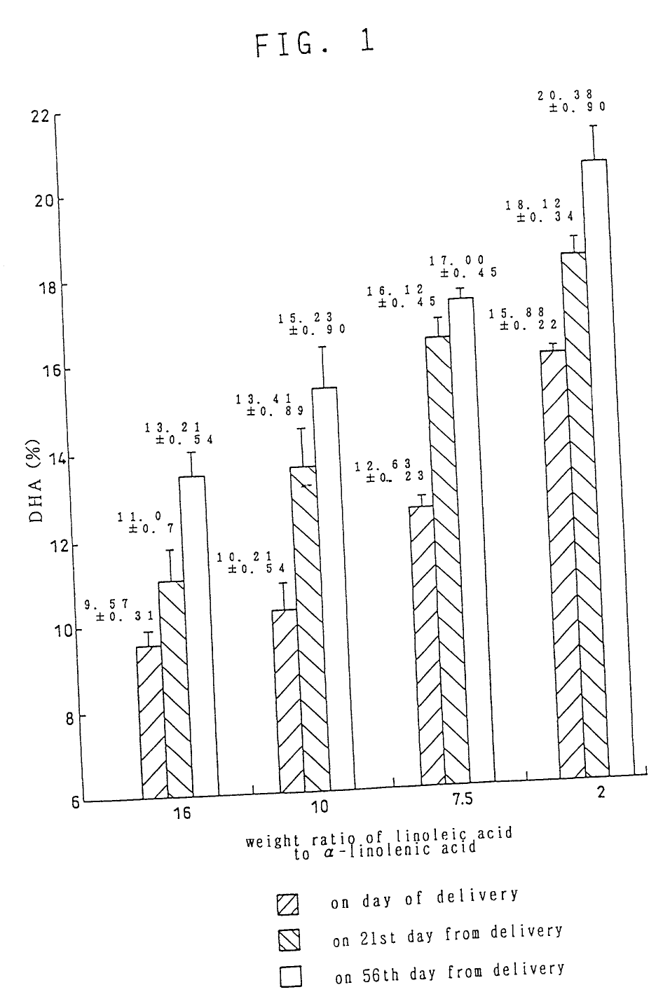

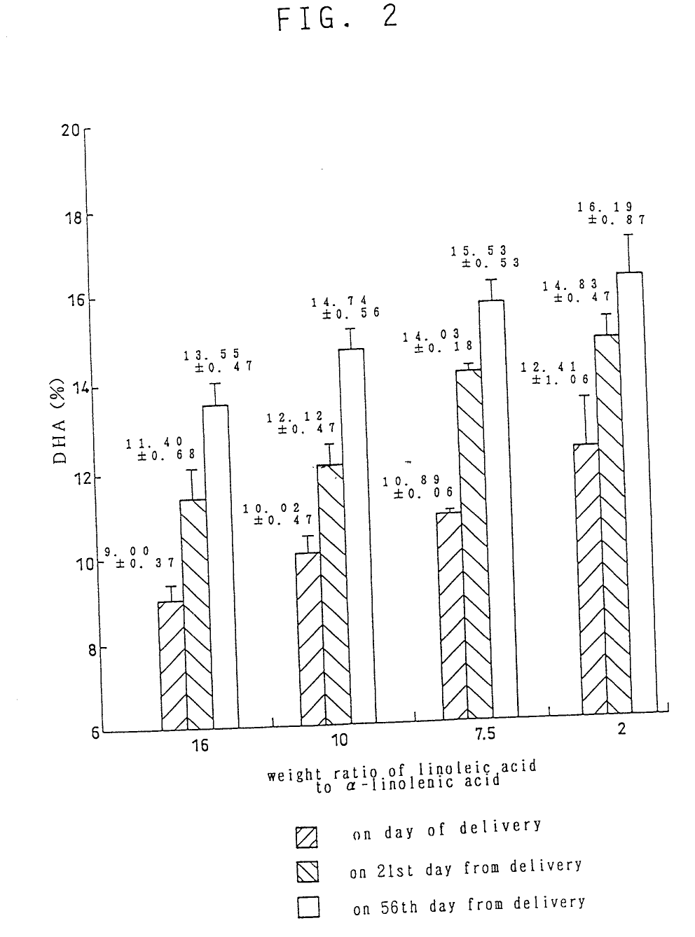

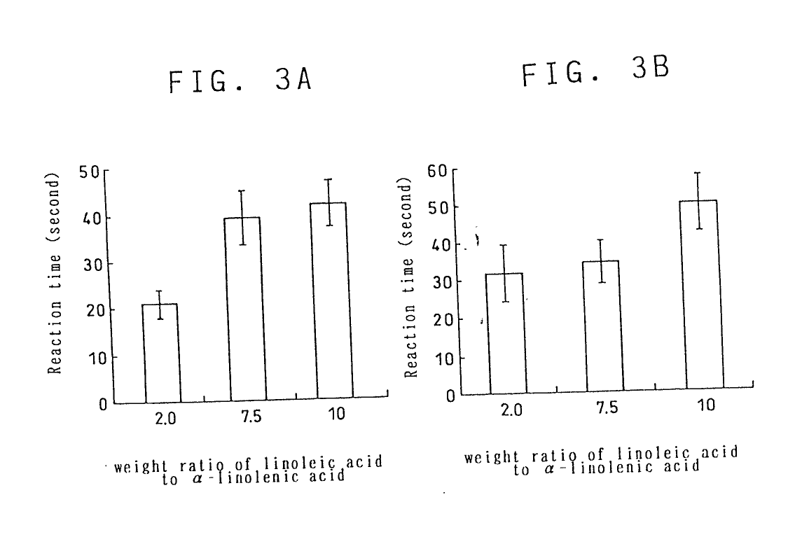

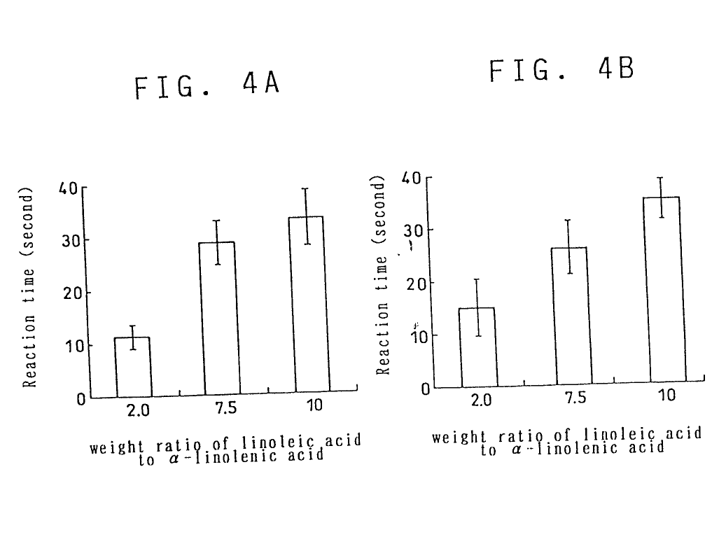

The results of cerebral fatty acid analysis, that is, changes in the DHA concentration in the brain are shown in FIGS. 1 and 2. Among the results of the Morris maze test, the results of cognitive ability (learning ability) tests are shown in FIG. 3 and the results of the memory power tests are shown in FIG. 4.

FIGS. 2, 3A and 4A are results for male young rats, while FIGS. 1, 3B and 4B are for female young rats.

In FIGS. 1 and 2, the ordinate represents the ratio of DHA to the total fatty acid in the brain of each young rat.

Morris maze test (learning ability and memory retention ability test)

The Morris maze test was used to evaluate both the learning ability and memory retention ability.

In this test, water was poured into a pool 160 cm in diameter and 50 cm deep. The water was clouded beforehand to zero visibility by adding starch. In the water, a footstool was placed at a position spaced 60 cm from the center of the pool in the one-o'clock direction. Using this pool, the above-mentioned eight-week-old male and female rats were subjected to the learning ability/memory retention ability test for 10 weeks.

In the first week, the rats were simply placed in the experiment room to let them accustomed to the experimental atmosphere. From the second week, the rats were divided into several groups, and the rats of each group were subjected to the learning test for four days. After the learning test, the rats were brought back to cages and held there for four weeks. Then, they were brought back into the pool and subjected to the test to evaluate their memory retention ability. The Morris maze test conditions this time were exactly the same as before.

In the learning ability/memory retention ability tests, the time periods until each rat found the stool in the clouded water and those required to remember where the stool were measured. (Needless to say, the water was so clouded that the rats could not see the stool.) For significant difference judgment in the Morris maze test, student t test, variance analysis (ANOVA), Dunnet's and Scheffe's tests were conducted, each for p<0.01 or p<0.05.

The results are shown in FIGS. 3 and 4. FIG. 3 shows the cognitive ability of each of the rats to which were given the feeds of which weight ratio of the linoleic acid to α-linolenic acid were 2.0, 7.5 and 10.0, that is, the time taken for each rat to find the stool in the clouded water, in the Morris maze test. FIG. 4 also shows their memory retention ability. In either case, the shorter the time, the higher the cognitive (learning) or memory retention ability.

Results

1. Cerebral fatty acid analysis

The DHA concentrations in the brain at delivery, and at 21st and 56th days after delivery were the highest for the rats that were given the feeds of which the ratio of linoleic acid to α-linolenic acid was 2. As the ratio increased from 2, the DHA concentration in the brain decreased.

2. Learning and memory retention abilities

As will be apparent from FIGS. 3 and 4, the feed of which the ratio of linoleic acid to α-linolenic acid was 2 achieved the best results. As the ratio increased from 2, the results got worse.

3. Results

Thus, it is apparent that the DHA concentration in the brain is the highest and the cognitive power and memory improve most remarkably when the feed of which the ratio of linoleic acid to α-linolenic acid was 2 was given to both babies and their mothers while they were pregnant.

By taking a composition comprising linoleic acid and α-linolenic acid mixed together in the above-defined ratio, the amount of DHA in the brain will increase apparently, so that it is possible to improve the cognitive ability and memory. Thus the present invention is very important for the food medical industry.

What is claimed is:

1. A higher unsaturated fatty acid composition comprising linoleic fatty acid (n-6, 18:2) and a linolenic fatty acid (n-3, 18:3), the weight ratio of said linoleic fatty acid to said α-linolenic fatty acid is 0.05 to 7.5.

2. The composition claimed in claim 1 wherein the weight ratio of said linoleic fatty acid to said a linolenic fatty acid is 0.05 to 2.0.

3. A food containing the higher unsaturated fatty acid composition claimed in claim 1 or 2.
